(19) United States
(12) Patent Application Publication
(10) Pub. No.: US 2026/0130995 A1
(43) Pub. Date: May 14, 2026

(54) METHODS AND COMPOSITIONS FOR TUMOR IMMUNOTHERAPY USING PATIENT-DERIVED IMMUNE CELL PRODUCTS AND IL-15 SUPERAGONISTS

(71) Applicant: Nant Holdings IP, LLC, Culver City, CA (US)

(72) Inventor: Patrick Soon-Shiong, Culver City, CA (US)

(73) Assignee: Nant Holdings IP, LLC, Culver City, CA (US)

(21) Appl. No.: 19/346,457

(22) Filed: Sep. 30, 2025

Related U.S. Application Data
(60) Provisional application No. 63/702,098, filed on Oct. 1, 2024.
(60) Provisional application No. 63/706,895, filed on Oct. 14, 2024.
(60) Provisional application No. 63/725,943, filed on Nov. 27, 2024.
(60) Provisional application No. 63/740,233, filed on Dec. 30, 2024.
(60) Provisional application No. 63/854,183, filed on Jul. 30, 2025.

(51) Int. Cl. A61K 40/15; A61K 38/20; A61K 40/11; A61K 40/19; A61K 40/31; A61P 35/00; C12N 5/0783

(52) U.S. Cl. CPC A61K 40/15; A61K 38/2086; A61K 40/11; A61K 40/19; A61K 40/31; A61P 35/00; C12N 5/0646; C12N 2501/2315

(57) ABSTRACT

Provided herein are methods, compositions, and kits for treating tumors by combining patient-derived immune effector cells with cytokine and therapeutic agents. In one aspect, apheresis material is processed to isolate at least two products selected from T cell, NK cell, and dendritic cell populations, which are formulated and administered to the patient. In another aspect, invariant natural killer T (iNKT) cells are expanded ex vivo using an IL-15:IL-15Rα complex and α-galactosylceramide delivered on a bacterial minicell nanoparticle, enabling scalable production of functional cells. Additional embodiments include tumor microenvironment preconditioning regimens and use of IL-15 superagonists to convert “cold” tumors into “hot” tumors.

### RELATED APPLICATIONS

This application claims priority to U.S. provisional applications with the Ser. No. 63/702,098, filed Oct. 1, 2024; Ser. No. 63/706,895, filed Oct. 14, 2024; Ser. No. 63/725,943, filed Nov. 27, 2024; Ser. No. 63/740,233, filed Dec. 30, 2024; and Ser. No. 63/854,183, filed Jul. 30, 2025. Each of these applications are incorporated by reference herein in its entirety.

### FIELD OF THE INVENTION

The field of the invention relates to composition and methods for use in immunotherapy.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Cancer immunotherapy has emerged as a transformative approach for the treatment of a variety of malignancies. Among the most successful modalities are checkpoint inhibitors that target immune checkpoints such as PD-1, PD-L1, and CTLA-4, thereby releasing inhibitory brakes on cytotoxic T cells. While checkpoint inhibitors have demonstrated durable responses in subsets of patients, the majority of tumors either fail to respond initially or acquire resistance during treatment. A key mechanism underlying this resistance is the loss or downregulation of major histocompatibility complex class I (MHC-I) molecules on tumor cells. Because MHC-I molecules are essential for antigen presentation to CD8+ T cells, their loss results in immune “cold” tumors that evade cytotoxic T cell surveillance. Patients with MHC-I negative tumors typically experience checkpoint inhibitor failure and disease progression.

Adoptive cellular therapies, such as chimeric antigen receptor (CAR)-T cells and CAR-NK cells, have also been developed to enhance tumor-specific immune responses. However, these therapies face limitations in solid tumors due to poor persistence, hostile tumor microenvironments, and mechanisms of immune evasion such as MHC loss, TGFβ-mediated immunosuppression, and lack of effective antigen presentation. Similarly, strategies employing dendritic cells (DCs) have shown promise for antigen presentation and immune priming, but DC-based therapies alone have not been sufficient to overcome resistance in MHC-deficient tumors.

Another avenue of investigation has been the use of natural killer (NK) cells, which are capable of killing MHC-I negative tumor cells through activating receptors such as NKG2D. Nevertheless, the clinical activity of NK cells has been limited by insufficient expansion, persistence, and functional exhaustion. Cytokine therapies, such as interleukin-2 (IL-2) or interleukin-15 (IL-15), have been explored to enhance NK and T cell function, but systemic cytokine administration often leads to toxicity and non-specific immune activation.

Invariant natural killer T (INKT) cells, also referred to as type I NKT cells, represent a distinct subset of T lymphocytes that bridge features of conventional αβ T cells and NK cells. iNKT cells express a semi-invariant T cell receptor (TCR), typically composed of an invariant Vα24-Jα18 TCR α-chain paired with a Vβ11 TCR β-chain in humans, and recognize lipid antigens presented by CD1d, a non-polymorphic antigen-presenting molecule. One of the best-characterized ligands for iNKT cells is α-galactosylceramide (α-GalCer), which binds CD1d and potently activates iNKT cells. Although iNKT cells comprise less than 1% of peripheral blood T cells in humans, they are highly conserved across species and play a critical role in immune regulation and tumor surveillance.

Upon activation, iNKT cells rapidly secrete large quantities of cytokines such as interferon-gamma (IFN-γ) and IL-4, which can act on both innate and adaptive immune effectors. Through this cytokine burst, INKT cells activate NK cells, dendritic cells, CD4+ helper T cells, and CD8+ cytotoxic T lymphocytes, thereby amplifying broad and coordinated antitumor responses. Importantly, iNKT-mediated immune responses are not restricted by classical MHC molecules, allowing recognition of lipid antigens without the limitations of MHC polymorphism. Because CD1d is monomorphic and expressed only on specific cell types, the use of iNKT cells in autologous or allogeneic settings reduces the risk of off-target effects and graft-versus-host disease, making them highly attractive as universal therapeutic agents.

However, the clinical translation of iNKT-based therapies is hindered by two major challenges. First, INKT cells exist at extremely low frequencies in human peripheral blood, typically less than 1% of total T cells. Second, conventional T cell expansion methods are poorly suited for iNKT cells, often producing suboptimal yields and impaired functionality. These limitations severely restrict the ability to generate therapeutic doses of iNKT cells for clinical applications.

Accordingly, there remains a significant unmet need for therapies that (i) overcome tumor immune evasion caused by MHC-I loss, (ii) convert “cold” tumors into “hot” tumors responsive to checkpoint inhibitors, (iii) expand and sustain effector immune cell populations including NK cells, T cells, dendritic cells, and iNKT cells, and (iv) provide robust ex vivo expansion methods for iNKT cells that preserve their functionality.

The present disclosure addresses these problems by providing integrated methods and compositions that combine patient-derived immune cell products, cytokine superagonists, and defined preconditioning regimens to generate durable and synergistic immune responses. In particular, the present disclosure leverages the inventors' unexpected finding that NK cell activation by an IL-15 superagonist induces interferon gamma (IFNγ) secretion at levels sufficient to restore MHC-I and MHC-II expression in tumors, thereby converting immune “cold” tumors into immune “hot” tumors and rescuing checkpoint inhibitor efficacy. Furthermore, this disclosure provides novel ex vivo expansion strategies for iNKT cells, enabling their clinical translation as universal immune effectors capable of coordinating innate and adaptive antitumor immunity.

### SUMMARY OF THE INVENTION

The present disclosure relates to compositions and methods for treating tumors, including tumors that are resistant to conventional immunotherapies. In certain aspects, the disclosure provides methods of treating a tumor in a patient in need thereof by collecting apheresis material from the patient, isolating at least two immune cell products therefrom, and administering a composition comprising the cell products back to the patient. The immune cell products include T cell products (such as γδ T cells, circulating tumor-educated lymphocytes (TELs), ex vivo educated TELs, γδ CAR-T cells, and αβ CAR-T cells), NK cell products (such as invariant natural killer T (INKT) cells, NK cells, memory-like cytokine enhanced NK (M-CENK) cells, ex vivo TEL INKT cells, CAR-NK cells, CAR-M-CENK cells, CAR-T cells, and CAR-INKT cells), and dendritic cell products (including educated dendritic cells). In some embodiments, two immune cell products are administered together, such as a T cell product and an NK cell product, a T cell product and a dendritic cell product, or an NK cell product and a dendritic cell product. In other embodiments, three immune cell products are combined in a therapeutic composition.

In another aspect, the present disclosure provides methods of expanding ex vivo invariant natural killer T (INKT) cells. In one embodiment, iNKT cells are isolated from a patient apheresis material intermediate (AMI) and exposed to an IL-15:IL-15Rα complex and a bacterial minicell-derived nanoparticle comprising α-galactosyl ceramide (αGC). In some embodiments, the INKT cells are isolated using anti-iNKT microbeads, for example microbeads specific for the T cell receptor α-chain Vα24-Jα18, and expanded in the presence of an IL-15:IL-15Rα complex. The expanded INKT cells may then be formulated for administration to a patient for therapeutic use. Pharmaceutical compositions and kits comprising iNKT cells, IL-15:IL-15Rα complexes, and αGC-loaded bacterial minicells are also provided.

In a further aspect, the present disclosure provides methods of preconditioning the tumor microenvironment prior to tumor resection or biopsy. Such methods include administration of therapeutic agents that modulate antigen presentation, inhibit TGFβ signaling, stimulate NK and T cell proliferation and activation, activate B cells, and/or enhance the development of tertiary lymphoid structures. Exemplary agents include class I Histone Deacetylase (HDAC) inhibitors (e.g., zabadinostat, etinostat, or nanatinostat), Protein Arginine N-Methyl Transferase 5 (PRMT5) inhibitors, Transforming Growth Factor beta (TGFβ) traps such as N-830 or HCW-9218, IL-15 or IL-15:IL-15Rα complexes such as N-803, B cell activators such as AT1965, and antibodies such as sotevtamab or anti-CTLA-4 antibodies. In certain embodiments, a defined combination regimen comprising Carboplatin, Nab-Paclitaxel, N-803, Sotevtamab, and Eflornithine is administered, with defined dosing schedules.

In yet another aspect, the present disclosure provides methods of treating tumors that are resistant to checkpoint inhibitor therapy. These methods comprise administering an IL-15 superagonist, such as N-803, to stimulate proliferation and activation of NK cells, thereby inducing NK cell-mediated killing of MHC class I-negative tumor cells and secretion of interferon gamma (IFNγ). This mechanism converts an immune “cold” tumor lacking MHC class I expression into an immune “hot” tumor expressing both MHC class I and class II molecules, restoring sensitivity of the tumor to checkpoint inhibitors. The IL-15 superagonist further stimulates CD8+ killer T cells, CD8+ memory T cells, and CD4+ helper T cells, thereby enhancing adaptive antitumor immunity and establishing durable immune memory. Such methods are particularly effective in treating non-small cell lung cancer (NSCLC), bladder cancer, melanoma, and head and neck squamous cell carcinoma.

Unexpectedly, the inventors discovered that combinations disclosed herein not only enhance the immediate cytotoxic activity of effector immune cells but also reprogram the tumor microenvironment in ways not previously anticipated. For example, administration of an IL-15 superagonist was found to drive NK cells to secrete IFNγ at levels sufficient to induce both MHC class I and MHC class II expression on tumor cells, a phenomenon not typically associated with NK activity. This effect results in a durable conversion of immune “cold” tumors into immune “hot” tumors, thereby restoring responsiveness to checkpoint inhibitor therapy in subjects who had previously failed such therapy. Additionally, the inclusion of eflornithine in combination regimens was unexpectedly found to synergize with N-803 by suppressing immunosuppressive metabolic pathways, further augmenting NK and T cell activity and enhancing the durability of clinical responses. These findings reveal unanticipated mechanisms of tumor sensitization and establish that the disclosed combinations achieve therapeutic outcomes not predictable from the known activity of the individual agents.

Collectively, the methods, compositions, and kits disclosed herein provide novel and synergistic approaches for immune-based treatment of cancer. By combining adoptive cellular therapy, ex vivo immune cell expansion, tumor microenvironment preconditioning, and cytokine-based immune reprogramming, the present disclosure addresses the major limitations of existing immunotherapies and provides durable clinical benefit, even in patients with checkpoint inhibitor-resistant tumors.

### BRIEF DESCRIPTION OF THE DRAWINGS

FIG. 1 depicts one embodiment of the Nant Cancer Vaccine.

FIG. 2 depicts one embodiment of the Nant Cancer Trifecta Vaccine: T cell pathway.

FIG. 3 depicts one embodiment of the Nant Cancer Trifecta Vaccine: NK and Dendritic cell pathway.

FIG. 4 depicts a successful transformation and Proliferation of Dendritic Cells from Apheresis Sample.

FIG. 5 depicts a successful transfection and education of Dendritic Cells with Tumor Associated Antigens (TAAs).

FIG. 6 depicts another embodiment of successful transfection and education of Dendritic Cells with Tumor Associated Antigens (TAAs).

FIG. 7 depicts another embodiment of successful transfection and education of Dendritic Cells with Tumor Associated Antigens (TAAs).

FIG. 8 depicts CA-125 levels for a Ovarian Cancer Stage 3 patient.

FIG. 9 depicts hematological data for the same patient.

FIG. 10 depicts a method of converting “cold” tumors into “hot” tumors.

FIG. 11 depicts a method of converting “cold” tumors into “hot” tumors.

FIG. 12 depicts TEL generation: cancer antigen stimulation of educated T cells.

### DETAILED DESCRIPTION

Embodiments of the present disclosure provides methods of treating a tumor in a patient by obtaining an apheresis material from the patient and isolating at least two immune cell products therefrom. The immune cell products may include: (1) T cell products, comprising γδ T cells, circulating TELs, ex vivo educated TELs, γδ CAR-T cells, and αβ CAR-T cells; (2) NK cell products, comprising iNKT cells, NK cells, memory-like cytokine-enhanced NK (M-ceNK) cells, ex vivo TEL INKT cells, CAR-NK cells, CAR-M-ceNK cells, CAR-T cells, and CAR-INKT cells; and (3) Dendritic cell products, comprising educated dendritic cells.

The isolated immune cell products may be expanded, engineered, or otherwise manipulated ex vivo before administration, or may be formulated directly for infusion. At least two, or preferably all three, of the T cell, NK cell, and dendritic cell products may be administered to the patient in a therapeutic composition to treat the tumor.

In certain embodiments, the NK cell product comprises iNKT cells, either native or engineered, which provide both direct cytotoxic activity and rapid cytokine release upon activation. The combination of iNKT cells with NK or T cell products yields synergistic immune responses, activating both innate and adaptive immunity within the tumor microenvironment.

In further embodiments, administration of the immune cell products is combined with an IL-15 superagonist, such as the pharmacokinetically stabilized IL-15:IL-15Rα fusion complex N-803 (Anktiva). IL-15 superagonists enhance proliferation and persistence of NK cells, CD8+ killer T cells, CD8+ memory T cells, and CD4+ helper T cells, while avoiding stimulation of Treg cells.

Ex Vivo Expansion of INKT Cells: In embodiments described herein, the present disclosure relates to improved methods and compositions for the ex vivo expansion of iNKT cells. INKT cells are a rare subset of T lymphocytes that express an invariant T cell receptor (TCR) α-chain, specifically Vα24-Jα18 in humans, and recognize glycolipid antigens presented by CD1d molecules. INKT cells are known to play pivotal roles in immune regulation, exhibiting both innate and adaptive features. Their capacity to secrete large amounts of cytokines upon activation, along with their cytotoxic activity and regulatory potential, renders them highly attractive candidates for immunotherapeutic strategies against cancer, infections, and autoimmune diseases. However, a major barrier to their therapeutic use is their extremely low frequency in peripheral blood, generally constituting less than 1% of total T cells. This necessitates the development of robust and scalable methods for their enrichment and ex vivo expansion.

Disclosed herein are methods for expanding iNKT cells from a patient-derived apheresis material intermediate (AMI). The method involves isolating iNKT cells from AMI, then exposing the isolated cells to a combination of an IL-15:IL-15 receptor alpha (IL-15Rα) complex and a bacterial minicell-derived nanoparticle carrying alpha-galactosyl ceramide (αGC). The IL-15:IL-15Rα complex acts as a potent stimulant for the proliferation and survival of iNKT cells, while αGC serves as a specific glycolipid antigen that activates the invariant TCR on iNKT cells. The use of bacterial minicell-derived nanoparticles facilitates the stable incorporation, protection, and presentation of αGC in a format that mimics natural antigen-presenting cells, thereby enhancing its immunostimulatory effect. This combination results in a synergistic activation and proliferation of iNKT cells under ex vivo conditions.

The isolation of iNKT cells from AMI may be achieved using antibody-conjugated microbeads that specifically bind the invariant TCR. Preferably, the microbeads are conjugated with antibodies against the human Vα24-Jα18 TCR α-chain. In certain embodiments, the microbeads are magnetic, allowing for rapid and efficient isolation via magnetic column chromatography. This technique yields a highly purified population of iNKT cells suitable for downstream expansion and clinical application. Alternative methods of INKT cell isolation are also envisioned by the inventors, for example wherein other iNKT-specific cell surface proteins are used to capture iNKT cells from the patient AMI.

In preferred embodiments, the IL-15:IL-15Rα complex used in the expansion process is N-803 (Anktiva), a superagonist comprising an IL-15 mutant bound to a dimeric IL-15Rα sushi domain fused to an IgG1 Fc. N-803 has demonstrated enhanced bioactivity and stability compared to native IL-15 and has been shown to improve the proliferation and effector function of iNKT cells in preclinical studies. Expanded iNKT cells typically express surface markers such as CD3 and CD56, confirming their identity and activation status. These expanded cells may then be formulated into a pharmaceutical preparation for therapeutic administration, either fresh or cryopreserved, in a dose suitable for a patient in need thereof. Alternatively, IL-15 or a stabilized derivative thereof, may be used. All of IL-15, IL-15:IL-15Rα, and derivatives thereof which activate immune cells comprising cell-surface expressed IL-15Rβγ are envisioned in embodiments of the invention.

In another embodiment, the method may be applied directly to the AMI without prior isolation of iNKT cells. In this approach, patient-derived AMI is exposed to the IL-15:IL-15Rα complex and αGC, optionally presented in a stabilized form. αGC may be chemically linked to a stabilizing protein such as albumin or streptavidin to improve its half-life and reduce degradation. Alternatively, αGC may be incorporated into bacterial minicell-derived nanoparticles to improve its delivery, presentation, and immunogenicity. The nanoparticles may be derived from non-pathogenic bacteria and engineered to include surface molecules that mimic antigen-presenting cells, thereby enhancing iNKT cell stimulation. These particles may display natural bacterial outer membrane structures and may optionally express ligands or antibodies for selective targeting.

In certain embodiments, pharmaceutical compositions are disclosed that include expanded iNKT cells derived from AMI, an IL-15:IL-15Rα complex (e.g., N-803), and bacterial minicell-derived nanoparticles carrying αGC. Such compositions may be administered to patients as cellular immunotherapies for the treatment of cancer or other immune-related diseases. Alternatively, kits are provided that contain all necessary components for iNKT expansion, including purified or AMI-derived INKT cells, cytokine complexes, nanoparticles containing αGC, and instructions for clinical or laboratory use. These kits facilitate point-of-care manufacturing or research applications.

Additionally, in certain embodiments, the compositions disclosed herein may be used to promote localized iNKT cell activation and proliferation at the site of a tumor. In such cases, bacterial minicell nanoparticles are engineered to display tumor-targeting moieties (e.g., antibodies or ligands specific for tumor-associated antigens) on their surface and contain αGC as an internal cargo. This design enables targeted delivery of the iNKT-activating glycolipid directly to the tumor microenvironment, where locally infused or systemically administered iNKT cells may be stimulated in situ. Co-administration or co-formulation with IL-15:IL-15Rα complex further supports proliferation and persistence of the INKT cells, enhancing the antitumor immune response while minimizing systemic toxicity.

Notably, the methods and compositions described in this disclosure provide a significant advance in the field of iNKT-based immunotherapy, offering practical, scalable, and clinically translatable approaches for iNKT enrichment, expansion, and activation.

The present disclosure further provides pharmaceutical compositions comprising expanded iNKT cells, IL-15:IL-15Rα complexes, and bacterial minicell-derived nanoparticles carrying αGC. Such compositions may be used ex vivo to expand iNKT cells or in vivo to stimulate activation and proliferation of iNKT cells at the site of a tumor.

In some embodiments, the bacterial minicell nanoparticle further comprises a targeting moiety expressed on the surface of the minicell and an intraparticle cargo comprising αGC, thereby enabling tumor-localized delivery.

The present disclosure also provides kits comprising one or more of: isolated iNKT cells from patient AMI, IL-15:IL-15Rα complexes, bacterial minicell nanoparticles with αGC, and directions for clinical use.

The present disclosure further provides methods for preconditioning the tumor microenvironment (TME) in vivo prior to tumor resection or biopsy. Preconditioning comprises administering to the individual one or more therapeutic agents selected from: (a) molecules that enhance expression of MHC-I and/or MHC-II peptides in antigen-presenting cells (e.g., class I HDAC inhibitors such as zabadinostat, etinostat, or nanatinostat, or PRMT5 inhibitors); (b) molecules that inhibit TGFβ activity (e.g., TGFβ traps such as N-830 or HCW-9218/TGFRt15-TGFRs); (c) molecules that stimulate NK cells, CD8+ T cells, CD4+ T cells, and/or memory T cells (e.g., IL-15 or IL-15:IL-15Rα complexes such as N-803); (d) molecules that activate B cells (e.g., AT1965); and (e) molecules that enhance development of tertiary lymphoid structures (e.g., anti-clusterin antibodies such as sotevtamab, or anti-CTLA-4 antibodies).

In certain embodiments, a defined pharmaceutical regimen is administered comprising carboplatin, nab-paclitaxel, N-803, sotevtamab, and eflornithine. Exemplary doses include nab-paclitaxel at 100 mg/m² weekly for three weeks of a four-week cycle, N-803 at 15 mg/kg, sotevtamab at 12 mg/kg weekly for at least two weeks, and eflornithine at 750 mg BID (bis in die, twice daily). Carboplatin may be administered once every four weeks for at least two cycles.

A particularly unexpected finding underlying the present disclosure is that administration of IL-15 superagonists, such as N-803, not only stimulates proliferation and activity of NK cells but also induces robust secretion of interferon-gamma (IFNγ). The NK cell derived IFNγ restores MHC-I and MHC-II expression in tumor cells, thereby converting MHC-deficient “cold” tumors into immunogenic “hot” tumors. This reprogramming restores responsiveness to checkpoint inhibitor therapy, including anti-PD-1, anti-PD-L1, and anti-CTLA-4 antibodies.

Thus, the present disclosure provides methods of treating “cold” immunotherapy resistant tumors by administering IL-15 superagonists to activate NK cells, kill MHC-I-negative tumor cells, induce IFNγ secretion, and restore adaptive T cell immunity. These methods are applicable to cancers such as non-small cell lung cancer (NSCLC), bladder cancer, melanoma, and head and neck squamous cell carcinoma.

In some embodiments, NK cell-mediated killing and IFNγ-driven MHC restoration induces durable immune memory, prolonging overall survival even in subjects who have failed prior checkpoint inhibitor or chemotherapy regimens.

### Trifecta Vaccine

In one aspect, the present disclosure provides methods for generating therapeutic immune cell products from a single apheresis collection. The method begins with obtaining an apheresis collection of white blood cells, which serves as the source material for the production of immune effector cells of both the innate and adaptive immune systems. In certain embodiments, whole blood may also be used, with white blood cells extracted using a Receptor Binding Site (RBS) antibody mixture. Preferably, a fresh, non-cryopreserved apheresis product is employed. The use of fresh material confers several advantages over cryopreserved products. Freshly collected cells maintain higher viability and reduced apoptosis compared to cells that have been frozen and thawed. In addition, fresh lymphocytes display superior proliferative capacity during ex vivo expansion and retain stronger functional activity, including cytotoxicity, antigen presentation, and cytokine production. The elimination of cryopreservation steps avoids the use of cryoprotectants such as dimethyl sulfoxide (DMSO), thereby reducing toxicity and product variability. Moreover, processing fresh material shortens the manufacturing timeline by permitting immediate initiation of therapeutic cell production.

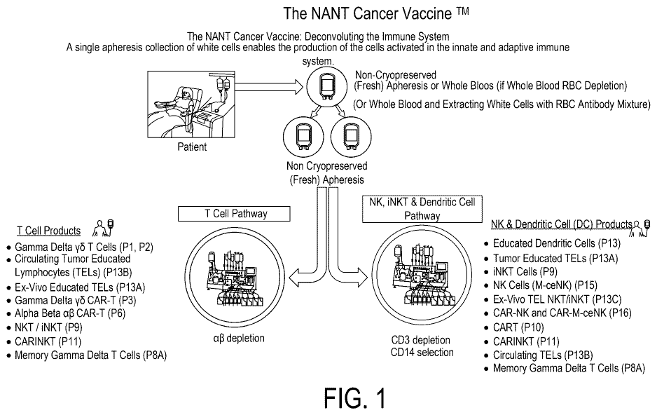

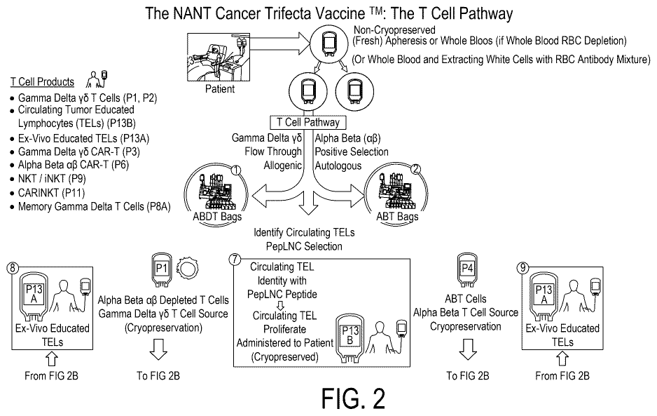

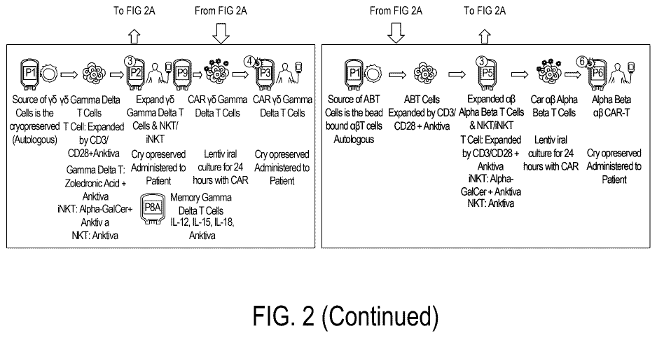

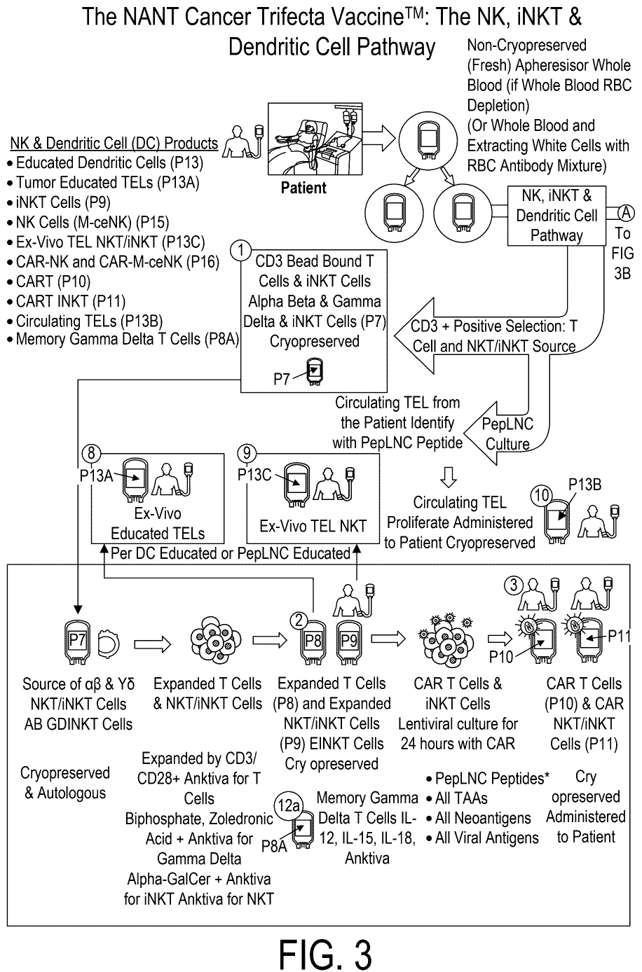

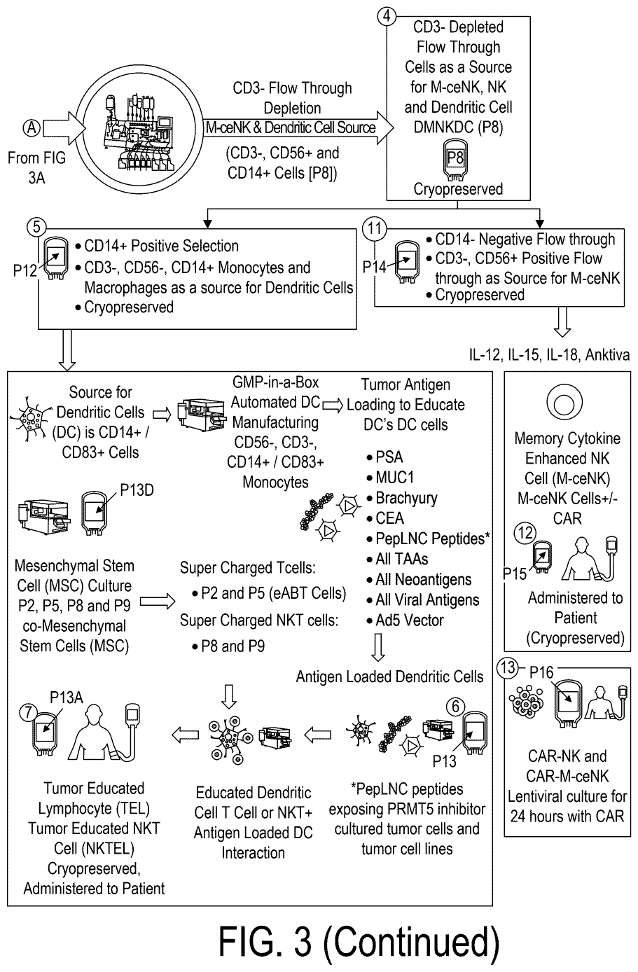

Following collection, the apheresis product is divided into separate portions for parallel processing pathways. In one pathway, the material is subjected to αβ depletion to enrich γδ T cells. T cells are broadly classified into αβ T cells, which constitute the majority of circulating T cells and recognize peptide antigens in an MHC-dependent manner, and γδ T cells, which represent a smaller subset and recognize antigens in an MHC-independent fashion. αβ depletion is performed to selectively remove αβ T cells, which are the primary mediators of graft-versus-host disease (GVHD) in allogeneic transplantation and adoptive transfer. The removal of αβ T cells thereby enriches for γδ T cells, thus enabling separation of those T cells that exhibit potent, broad-spectrum anti-tumor activity. NK cells, which remain after αβ depletion, further contribute innate cytotoxicity. The resulting γδ T cell and NK cell enriched product can be infused into patients to target tumors with enhanced safety and reduced toxicity compared to unmodified T cell populations. In certain embodiments, αβ depletion is achieved by immunomagnetic bead-based separation using antibodies against TCRαβ, or by flow cytometry based sorting. The unbound fraction, negative for TCRαβ, contains the desired γδ T cell and NK cell populations. The T cell pathway is further disclosed in FIGS. 1-3.

In a second pathway, the apheresis product is processed to generate NK cells through CD3 depletion. CD3 is a marker expressed on all mature T cells but is absent on NK cells. Selective removal of CD3+ cells eliminate T cells, while enriching for CD3−, CD56+, and CD14− NK cells. These NK cells retain their natural ability to lyse malignant cells through natural cytotoxicity receptors and killer immunoglobulin-like receptors and can also mediate antibody-dependent cellular cytotoxicity via CD16. Because NK cells are not restricted by MHC recognition, they are capable of targeting diverse tumor types. CD3 depletion may be carried out by immunomagnetic bead-based separation using anti-CD3 antibodies. The CD3− negative fraction obtained from this process provides an enriched NK cell population, which may be further expanded or activated ex vivo using cytokines such as IL-2 and/or IL-15. The NK cell population may be further modified through the addition of a CAR, as further disclosed in WO-2020/096646, WO-2019/226708, WO-2019/177986, WO-2020/091868, WO-2020/028656, WO-2021/154218, WO-2020/091869, all of which are incorporated by reference in its entirety. The NK cell population may also be modified as described in WO-2022/187207 or WO-2025/038711 (incorporated by reference herein) to produce Memory-like cytokine enhanced NK (M-ceNK) cells. In general, M-ceNK cells refer to NK cells that have been preactivated or primed with one or more of IL-12, IL-15, and IL-18, to induce long-lasting functional changes resembling immunological memory. M-ceNK cells exhibit enhanced cytotoxicity, increased proliferation, and augmented cytokine secretion, including IFNγ, upon restimulation with tumor or viral targets. Unlike conventional NK cells, which are typically short-lived and rapidly exhausted, M-ceNK cells display sustained effector activity and persistence in vivo, making them even more suitable for cancer immunotherapy and infectious disease treatment.

The NK cell product as prepared and described above complements the T cell pathway by engaging innate immune mechanisms to enhance anti-tumor efficacy. The NK cell pathway is further disclosed in FIGS. 1-3.

In a third pathway, the apheresis product is processed to generate dendritic cells (DCs) through CD14 selection and/or CD83 selection. CD14 is a surface receptor expressed on monocytes and macrophages, which serve as precursors to dendritic cells. Selection of CD14+ and/or CD83+ monocytes provide a highly enriched starting population that may be differentiated into immature dendritic cells by culturing with granulocyte-macrophage colony-stimulating factor (GM-CSF) and IL-4. Subsequent maturation with factors such as tumor necrosis factor-alpha (TNFα), IL-1β, or prostaglandin E2 (PGE2) produces mature dendritic cells with potent antigen-presenting capability. These dendritic cells may be further loaded with tumor antigens, tumor lysates, or nucleic acids encoding tumor-associated antigens, thereby enabling presentation of tumor-specific epitopes. Tumor antigens loaded to educate the DC's comprise PSA, MUC1, Brachyury, CEA, PepLNC Peptides, TAAs, neoantigens, and/or viral antigens. The resulting dendritic cell product functions as a therapeutic vaccine, activating cytotoxic and helper T lymphocytes as well as NK cells against tumor cells. The dendritic cell pathway is further disclosed in FIGS. 1-3.

By dividing a single fresh apheresis collection into these parallel processing pathways, the present methods enable the simultaneous generation of T cell enriched products, NK cell enriched products, and dendritic cell products. These products work synergistically to form a multifaceted therapeutic platform designed to maximize anti-tumor efficacy.

Isolation of iNKT Cells

Invariant natural killer T (INKT) cells can be isolated from human blood using antibody-conjugated microbeads through a process known as magnetic-activated cell sorting (MACS), a technology developed and widely used in platforms such as those provided by Miltenyi Biotec. The isolation process begins with the collection of peripheral blood, from which leukapheresis products (heterogeneous mixture including red blood cells, platelets, plasma, and various immune cells) are separated. The leukapheresis product is further processed to deplete red blood cells and remove plasma. The remaining fraction, which includes T cells, NK cells, and iNKT cells, is concentrated to arrive at the Apheresis Material Intermediate (AMI). iNKT cells are characterized by their expression of a semi-invariant T cell receptor (TCR) composed of a Vα24-Jα18 α-chain, which is highly conserved in humans and serves as a specific surface marker for identification and isolation.

To selectively capture iNKT cells, the AMIs are incubated with magnetic microbeads conjugated to monoclonal antibodies that specifically recognize the Vα24-Jα18 TCR. These microbeads are superparamagnetic and can be manipulated using a magnetic field without retaining residual magnetism once the field is removed. Following antibody binding, the cell-bead suspension is passed through a MACS column situated within a magnetic separator. Cells labeled with the anti-Vα24-Jα18 microbeads, i.e., the iNKT cells, are retained in the column while the unlabeled cells flow through and are discarded or collected separately. After sufficient washing to remove non-specifically bound cells, the column is removed from the magnetic field, and the retained iNKT cells are eluted using a buffer.

This method enables the isolation of iNKT cells with high specificity and minimal impact on cell viability and function. The isolated iNKT cells are then expanded by exposing to a combination of an IL-15:IL-15 receptor alpha (IL-15:IL-15Rα) complex and a bacterial minicell-derived nanoparticle carrying alpha-galactosyl ceramide (αGC).

### IL-15:IL-15 Receptor Alpha (IL-15:IL-15Rα) Complex

In a particularly favored embodiment, the IL-15:IL-15Rα complex is nogapendekin alfa-imbakicept (NAI), also known as N-803, ALT-803, Anktiva, or IL-15N72D:1L-15RαSu/IgG1, which is an IL-15-based immunostimulatory protein complex comprising two protein subunits of a human IL-15 variant associated with high affinity to a dimeric human IL-15 receptor α (IL-15Rα) sushi domain/human IgG1 Fc fusion protein (J Immunol (2009) 183:3598-3607). The IL-15 variant is a 114 amino acid polypeptide comprising the mature human IL-15 cytokine sequence, with an asparagine to aspartate substitution at position 72 of helix C (N72D). The human IL-15Rα sushi domain/human IgG1 Fc fusion protein comprises the sushi domain of the human IL-15 receptor α subunit (IL-15Rα) (amino acids 1-65 of the mature human IL-15Rα protein) linked to the human IgG1 CH2-CH3 region containing the Fc domain (232 amino acids). U.S. Pat. No. 9,328,159, which describes NAI, is incorporated herein by reference in its entirety.

Most typically, the N-803 complex may have a molecular weight of approximately 114 kDa and will advantageously exhibit an extended serum half-life compared to native IL-15 due to the Fc fusion. Among further benefits, it should also be noted that N-803 may stimulate proliferation and/or activation of INKT cells. Moreover, N-803 will also induce production of cytokines like IFNγ. Suitable variants of N-803 may include complexes with different IL-15 mutations, such as IL-15N72A. The Fc portion may be derived from other IgG subtypes. Linker sequences between the components may be modified. The ratio of IL-15 to IL-15Rα may be altered. Exemplary sequences for suitable N-803 and analogs are shown in SEQ ID NO:1-4, of U.S. Patent Application No. 63/768,448, which is incorporated by reference, and which forms an express part of this disclosure. Other suitable IL-15 or IL-15:IL-15Rα constructs may comprise the single chain constructs of U.S. Pat. No. 11,401,324, or the multi-chain constructs of U.S. Pat. No. 11,518,792.

### Bacterial Minicells

Bacterial minicells are anucleate, nano-sized particles of approximately 400 nanometers in diameter, which are generated by genetically engineering bacteria such as Salmonella enterica to delete genes controlling normal cell division (e.g., minCDE), thereby derepressing polar division sites and causing aberrant fission. These minicells retain intact inner and outer membranes, a cytoplasmic compartment, and surface lipopolysaccharides (LPS), but lack chromosomal DNA and cannot replicate. Their structural integrity and biocompatibility make them an ideal platform for therapeutic delivery. Minicells can be loaded with various payloads, including cytotoxic chemotherapeutic agents, small interfering RNAs (siRNAs), short hairpin RNAs (shRNAs), and other molecular cargos such as glycolipids. Once loaded, minicells are functionalized with bispecific antibodies (BsAbs) that allow for targeted delivery: one antibody arm binds to the O-polysaccharide on the minicell surface, while the other binds to an antigen. In contemplated embodiments, the antigen may be a tumor-specific antigen.

Following intravenous administration, the large size of minicells prevents extravasation into healthy tissues, whose vascular endothelium is tightly sealed. However, tumor vasculature is structurally disorganized and leaky, with fenestrations that can exceed several hundred nanometers. Minicells passively accumulate in tumors via these openings and bind to target receptors via their BsAbs. Upon binding, they are internalized by tumor cells through receptor-mediated endocytosis, trafficked to lysosomes, and degraded. This results in the intracellular release of the therapeutic payload, whether cytotoxic drug or RNAi molecules, leading to apoptosis or gene silencing within the tumor cells. Minicells loaded with siRNAs against essential cell cycle genes like PLK1, CDK1, or MDR1 have demonstrated potent antitumor effects in vitro and in xenograft models, including reversal of multidrug resistance when used in sequential treatments with drug-loaded minicells.

Beyond direct tumor cytotoxicity, minicells can also trigger an anti-tumor immune response. Circulating minicells that do not enter the tumor are rapidly taken up by professional antigen-presenting cells (APCs) such as macrophages and dendritic cells, which recognize pathogen-associated molecular patterns (PAMPs) like LPS. This leads to APC activation, maturation, and migration to lymph nodes, where they present tumor antigens derived from dying cancer cells to T cells. This in turn primes CD8⁺ cytotoxic T cells and generates long-term immunological memory. This dual action, targeted cytotoxic delivery and immune system engagement, enables minicells to overcome tumor heterogeneity, immune suppression, and drug resistance. Minicells can be lyophilized for long-term storage, reconstituted on demand, and easily integrated into standard oncology workflows. As a multifunctional, tumor-targeted nanoparticle platform, bacterial minicells represent a unique approach to both drug delivery and cancer immunotherapy. Bacterial minicells are further disclosed in MacDiarmid J A, et al “Sequential treatment of drug-resistant tumors with targeted minicells containing siRNA or a cytotoxic drug”, Nat Biotechnol., 2009 July; 27 (7): 643-51, PMID: 19561595, and Brahmbhatt H et al “Bacterial minicells to the rescue: cyto-Immunotherapy for the treatment of late stage cancers with minimal to no toxicity”, Microb Biotechnol., 2022 January; 15 (1): 91-94, PMID: 34665932; PMCID: PMC8719834.

### Preconditioning TME

The present disclosure provides methods for preconditioning a TME in vivo prior to tumor biopsy or resection. In certain embodiments, the method comprises administering to an individual one or more agents selected from: (1) molecules that enhance the expression of MHCI and/or MHCII-bound peptides in antigen-presenting cells, (2) molecules that inhibit TGFβ activity, (3) molecules that stimulate proliferation and/or activation of NK cells, CD8⁺ T cells, CD4⁺ T cells, and/or memory T cells, (4) molecules that activate B cells, and (5) molecules that enhance the development of tertiary lymphoid structures. Representative embodiments include: class I HDAC inhibitors (e.g., zabadinostat, etinostat, nanatinostat) or PRMT5 inhibitors to increase antigen presentation, TGFβ traps (e.g., N-830, HCW-9218) to relieve immunosuppression, IL-15 superagonists (e.g., N-803) to expand NK and T-cell subsets, AT1965 to activate B cells, and antibodies that promote TLS formation, including anti-clusterin (e.g., Sotevtamab) and anti-CTLA-4 antibodies.

In one embodiment, the disclosure provides a defined multimodal regimen comprising Carboplatin, Nab-Paclitaxel, N-803, Sotevtamab, and Eflornithine. The inventors unexpectedly found that combination simultaneously reduces tumor bulk, enhances immune priming, remodels the stromal niche, and alters tumor metabolism to favor immune activation.

The present disclosure provides explicit preconditioning of the tumor microenvironment prior to resection. Neoadjuvant therapy is traditionally used to shrink tumors before surgery, often focusing on cytoreduction with chemotherapy or single immunotherapies. On the other hand, in this present disclosure, the therapeutic intent is not only tumor shrinkage but immune landscaping, such as enhancing antigen presentation, fostering tertiary lymphoid structures, and priming effector/memory lymphocytes before resection or biopsy. This shifts the paradigm from purely cytotoxic neoadjuvant treatment to a deliberate immune education strategy in vivo.

The combination of epigenetic antigen-presentation enhancers with TGF-β inhibition for the purpose of preconditioning the TME prior to resection or biopsy of a tumor provides surprising benefits. The combined use of PRMT5/HDAC inhibitors to upregulate MHC I/II peptide presentation, paired with TGF-β blockade to relieve suppression, creates a synergistic environment in which antigens are not only more visible but also immunologically actionable. Moreover, IL-15 superagonists like N-803 are currently being used for metastatic disease or combination checkpoint therapy, but its use in pre-surgical regimens is not known. In this case, using N-803 during neoadjuvant therapy expands NK and T-cell populations in situ, creating a pre-armed immune system at the time of surgery. The inventors contemplate that this helps in residual micrometastatic disease control and long-term immune memory post-resection.

Furthermore, incorporating an anti-clusterin antibody (Sotevtamab) to promote Tertiary Lymphoid Structures (TLS) formation represents a novel strategy. TLS formation is correlated with prognosis in several tumors but is rarely a deliberate therapeutic goal. The present disclosure that a neoadjuvant cocktail could reorganize immune architecture locally to sustain ongoing antitumor immunity is not taught in prior regimens.

Similarly, Eflornithine (DFMO) is classically studied in parasitology and some cancer metabolic contexts, but not standard in neoadjuvant chemo-immunotherapy. Its inclusion in the presently disclosed compositions and methods reflects recognition that polyamine metabolism intersects with immune suppression in the tumor microenvironment. Combining DFMO with immunostimulatory agents and TLS inducers is an unexpected immunometabolic angle.

In summary, the presently disclosed regimen reframes neoadjuvant therapy as an immune preconditioning protocol, where chemotherapy is only one component. The uniqueness lies in the synergistic orchestration of epigenetic, checkpoint/TGF-β, cytokine, metabolic, and TLS-inducing interventions to remodel the tumor microenvironment into a highly immunogenic state before surgery, which has not been currently achieved in standard oncology practice.

### Treating a Cold Tumor

The present disclosure further relates to methods and compositions for enhancing anti-tumor immunity by restoring immune recognition in tumors that evade checkpoint inhibitor therapy through loss of MHC-I expression. The approach leverages IL-15 superagonists (e.g., Anktiva) to stimulate NK cell activity, thereby converting MHC-negative “cold” tumors into MHC-positive “hot” tumors and rescuing checkpoint inhibitor responsiveness.

In normal immune surveillance, tumor antigens are taken up by dendritic cells (DCs). DCs present these antigens via MHC-I and MHC-II complexes, activating CD8+ cytotoxic T lymphocytes (CTLs), CD4+ helper T cells, and generating long-lived CD8+ memory T cells. This process, the DC priming of T cells, produces tumor-educated lymphocytes (TELs) capable of recognizing and eliminating tumor cells.

Tumor progression and resistance to checkpoint blockade is frequently associated with loss of MHC-I expression. Initially, normal tissues are HLA-positive; as tumors evolve, they become heterogeneous (mixed HLA+ and HLA− populations), and ultimately fully HLA−. Loss of MHC-I creates a “cold tumor” phenotype characterized by evasion of CD8+ T cells, immune dormancy, and eventual metastatic progression. Checkpoint inhibitors (e.g., anti-PD-1 antibodies) enhance CTL activity against MHC-positive tumors. However, when tumors lose MHC-I, CD8+ killer T cells cannot engage the target, rendering checkpoint inhibitors ineffective. Thus, MHC-I loss is a central mechanism of therapeutic resistance.

The inventors have now shown that unlike CTLs, NK cells selectively recognize and kill MHC-negative tumor cells. Therefore, MHC-I negative “cold tumors” across diverse tumor types represent a universal NK cell target. IL-15 superagonists such as Anktiva expand and activate NK cells, enabling tumor cell clearance in checkpoint inhibitor, chemotherapy, and BCG-refractory settings. Unexpectedly, it was found that NK cell activation does more than directly kill MHC-tumor cells. NK cell-mediated killing is accompanied by IFN-γ secretion, which re-induces MHC-I and MHC-II expression on tumor cells. This reprogramming function of NK cells was not predictable from prior knowledge, where NK cells were regarded primarily as short-lived cytolytic effectors.

NK cell-mediated lysis of MHC-I negative tumor cells involves NK cell secretion of IFNγ. IFNγ reactivates MHC-I and MHC-II expression, effectively converting cold tumors into hot tumors. This process restores tumor antigen presentation and re-engages CD4+ helper and CD8+ killer T cells. This conversion of a cold, therapy-resistant tumor into a hot, immunologically visible tumor is surprising because MHC loss was previously viewed as a terminal escape mechanism. The ability to reverse this phenotype establishes a new therapeutic paradigm.

By converting MHC− tumors into MHC+ tumors, IFNγ secretion enables checkpoint inhibitors to regain efficacy. Thus, NK cell activation not only directly reduces tumor burden but also restores the adaptive immune arm, rescuing checkpoint blockade therapy. Thus, the inventors found that checkpoint inhibitor failure due to MHC loss is not permanent; it can be reversed through NK/IL-15 driven IFNγ activity.

Both NK cells and T cells express IL-15 receptors. Anktiva, an IL-15/IL-15Rα superagonist complex, binds to the IL-15 receptor, leading to robust proliferation of NK cells, CD8+ killer T cells, and CD8+ memory T cells. This expands the effector and memory compartments necessary for durable tumor remission. Notably, treatment with Anktiva leads not only to NK expansion but also to durable CD8+ memory formation, overcoming the transient nature of NK-based therapies.

Anktiva mimics the function of activated dendritic cells by delivering IL-15/IL-15Rα signaling to proliferate NK and T cell subsets. This dual action (direct NK-mediated killing of cold tumors and restoration of T cell immunity) provides a universal immunotherapy platform across tumor types.

Thus, through NK cell-driven conversion of cold tumors to hot tumors, Anktiva reactivates MHC-I expression, restores checkpoint inhibitor sensitivity, expands CD8+ effector and memory T cells, and prolongs overall survival. This establishes a broadly applicable therapeutic strategy to overcome tumor immune evasion and resistance. Accordingly, the present disclosure reveals that MHC loss—a universal resistance mechanism to checkpoint blockade, chemotherapy, and BCG immunotherapy—is not irreversible, but can be overcome through NK/IL-15 activation. This was unexpected and represents a fundamental advance in cancer immunotherapy.

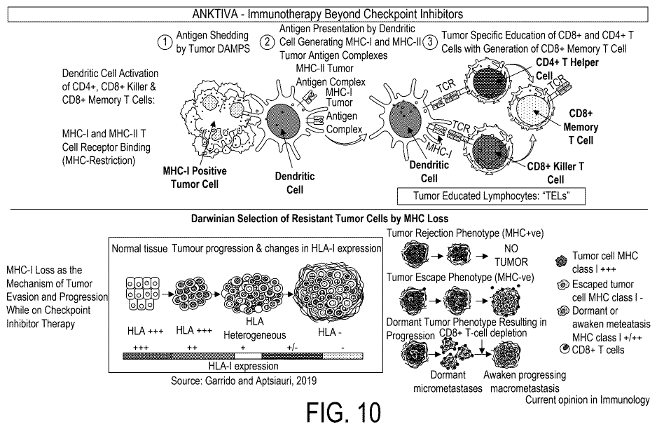

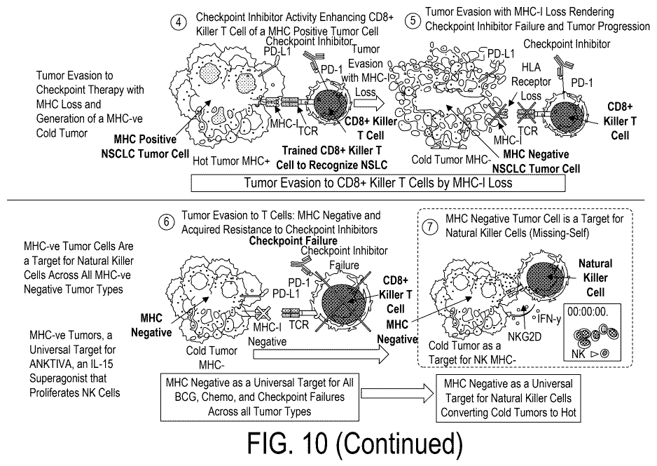

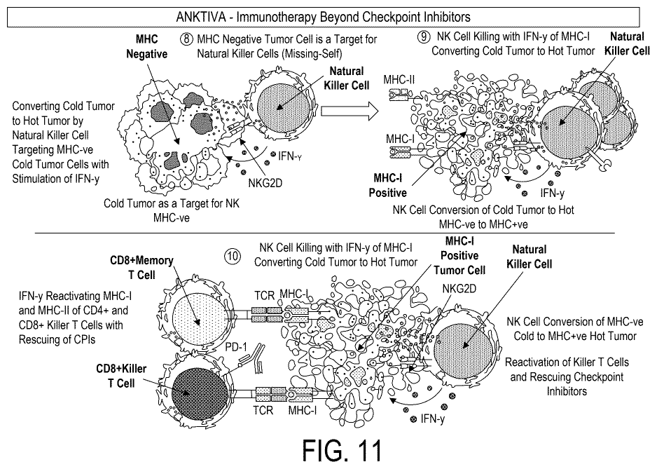

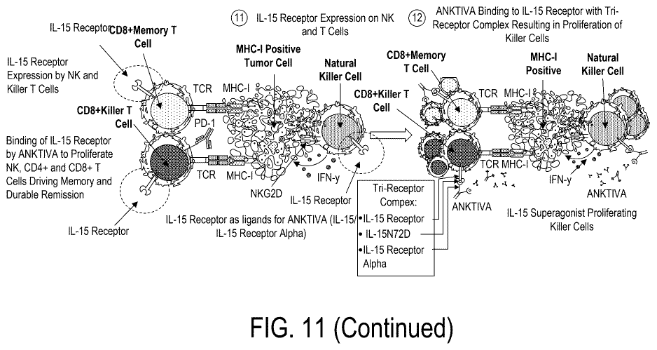

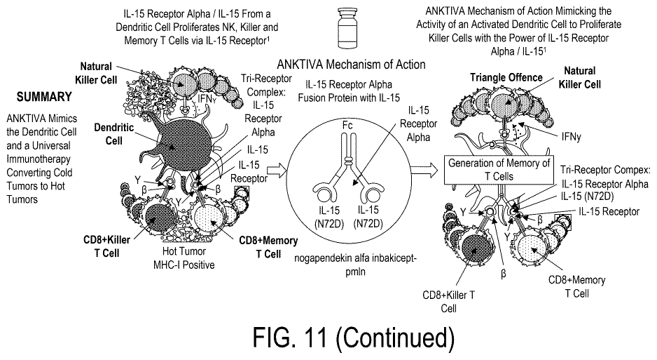

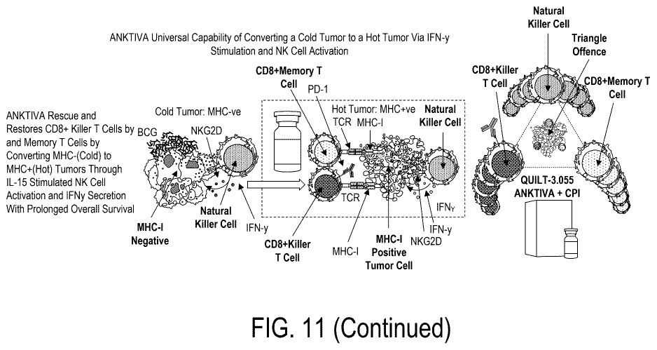

These methods are further described and shown in FIGS. 10-11.

Embodiments of the present disclosure are further described in the following examples. It should be understood that these examples are illustrative and should not be construed as being limiting in any way.

### Example 1

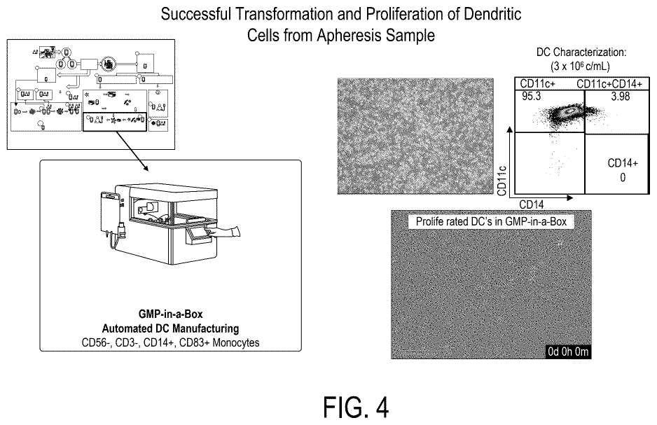

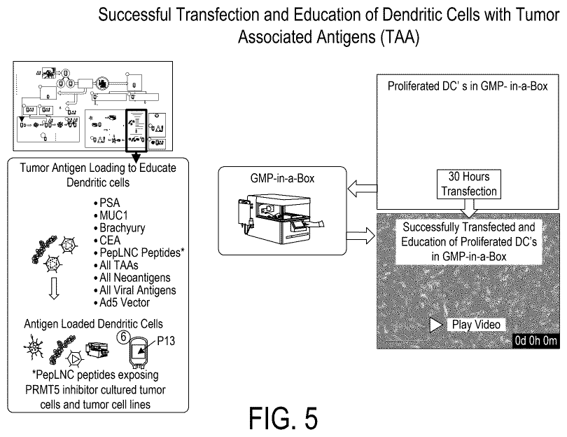

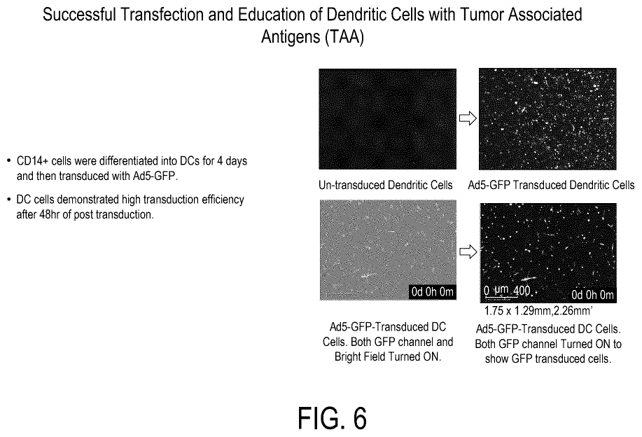

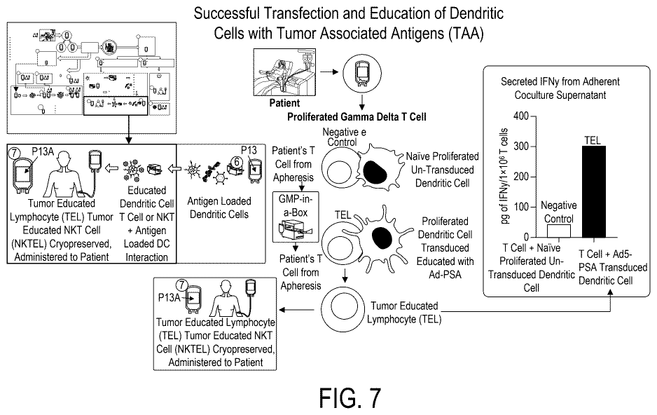

FIGS. 4-7 demonstrates the feasibility of ex vivo differentiation, genetic modification, and functional utilization of dendritic cells (DCs) from apheresis sample for tumor antigen presentation and subsequent T-cell education. This illustrates the successful transfection and education of Dendritic Cells with Tumor Associated Antigens (TAA).

Cellular Source and Separation: Fresh non-cryopreserved apheresis products were subjected to selective depletion and enrichment steps, including CD3 depletion, CD14 selection, and αβ depletion. Fractions were allocated to generate multiple immune cell products: αβ T cells, γδ T cells, NK cells, iNKT cells, and CD14⁺ monocytes (for DC differentiation).

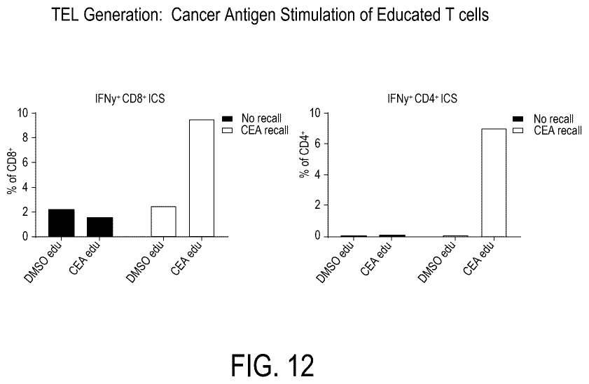

T Cell Pathway involved multiple techniques. The αβ T cells were obtained by expansion ex vivo using CD3/CD28 stimulation and Anktiva (N-803), with subsets subjected to lentiviral CAR transduction. The γδ T cells were obtained by expansion using zoledronic acid plus Anktiva; subsets were also CAR-transduced. The memory γδ T cells were obtained by inducing with IL-12, IL-15, IL-18, and Anktiva to enhance persistence. The TEL were generated by co-culturing of patient T cells with autologous antigen-loaded DCs, as shown in FIG. 12.

NK, INKT, and DC Pathway also involved multiple techniques. The NK/M-ceNK cells were derived from CD56⁺ fractions. M-ceNK was generated with cytokine cocktail (IL-12, IL-15, IL-18) for enhanced function and persistence. The iNKT cells were expanded using α-GalCer+Anktiva. The DCs were obtained from CD14⁺ monocytes differentiated into CD83⁺ DCs using GMP-in-a-Box automated culture.

The DCs were transduced with adenoviral vectors encoding GFP (Ad5-GFP) or tumor-associated antigens (Ad5-PSA, MUC1, CEA, Brachyury, PepLNC-derived peptides, or neoantigens). Antigen loading was confirmed by GFP fluorescence and downstream T-cell activation assays.

Functional Assays: Co-culture assays with patient-derived T cells were used to assess DC-mediated education. Supernatants were analyzed for IFNγ secretion as a marker of Th1 polarization and cytotoxic activation. Negative controls consisted of naïve or untransduced DCs co-cultured with T cells.

Based on the above experiments, the inventors were able to generate diverse immune cell products from a single apheresis. From a single apheresis, multiple immune subsets were successfully expanded and/or engineered, including αβ CAR-T, γδ CAR-T, NK, INKT, and TEL populations. Cytokine-conditioned memory subsets (γδT and NK) demonstrated enhanced expansion and potential for persistence.

CD14⁺ monocytes differentiated into DCs within four days, achieving CD83⁺ maturation markers in GMP-in-a-Box cultures. Ad5-GFP transduction yielded high efficiency within 48 hours, validating viral vector delivery to DCs.

Antigen-loaded DCs (e.g., Ad5-PSA-transduced) successfully induced TELs and NKTELs when co-cultured with autologous T cells. Functional readouts demonstrated robust IFNγ secretion in co-culture supernatants, consistent with antigen-specific activation and Th1 polarization. Negative controls (naïve DCs) did not elicit IFNγ, confirming specificity of the immune education.

TELs, NKTELs, CAR-T cells, γδ T cells, and M-ceNK were all cryopreserved post-expansion, establishing a pipeline for reinfusion into patients.

These findings demonstrate the feasibility of a multi-cellular, ex vivo immunotherapy platform capable of generating a comprehensive anti-tumor response. The efficient differentiation and viral transduction of DCs establish a reliable system for presenting a wide array of tumor-associated antigens, including classical TAAs, neoantigens, and viral epitopes. The ability of antigen-loaded DCs to educate T cells, confirmed by IFNγ secretion, underscores their capacity to prime tumor-reactive effector populations.

Mechanistically, IFNγ secretion indicates robust Th1 polarization, essential for cytotoxic function, macrophage activation, and recruitment of additional immune effectors. TELs and NKTELs generated ex vivo represent a highly personalized product, combining the specificity of antigen-driven T cells with the durability of adoptive transfer.

The integration of adaptive (αβ CAR-T, TELs), innate (NK, γδ T), and bridging (DCs, iNKT) components addresses major limitations of monotherapies. Tumor antigen escape is countered by redundancy across recognition pathways; immune exhaustion is mitigated by memory-like subsets conditioned with cytokine cocktails. Finally therapeutic durability is enhanced by cryopreservation and potential for serial dosing.

While the current literature treats CAR-T, NK, γδ T, and DC vaccines as separate manufacturing streams requiring different starting materials or separate collections, the inventors have unexpectedly demonstrated an integrated, single-apheresis pipeline that reproducibly yields multiple distinct, clinically relevant products (αβ CAR-T, γδ T, NK/M-ceNK, INKT, antigen-loaded DCs, and TELs). The presently disclosed methods notably overcomes logistical and biological separation barriers (cell yields, cross-contamination, differing culture conditions) that normally force independent processes.

The Trifecta Vaccine disclosed herein generates multiple antigen-specific and innate-like effector cell products from a single apheresis. By integrating dendritic cell-mediated antigen presentation, CAR engineering, cytokine-enhanced memory subsets, and ex vivo education of T cells, this platform aims to elicit a durable, redundant, and highly personalized anti-tumor immune response.

### Example 2

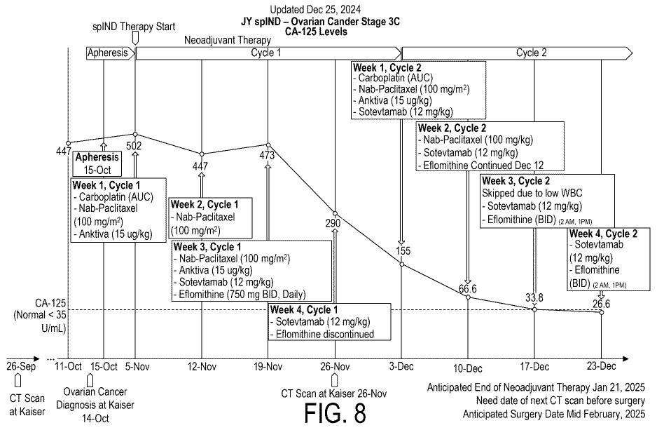

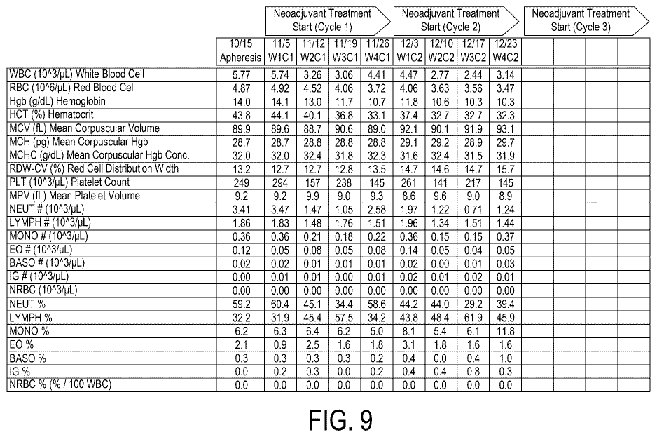

FIGS. 8-9 describe the treatment of a patient with stage 3 ovarian cancer using a neoadjuvant therapeutic regimen that included Carboplatin, Nab-Paclitaxel, Anktiva (N-803), Sotevtamab, and optionally Eflornithine.

The treatment comprised several cycles. Cycle 1 began with Carboplatin, Nab-Paclitaxel, and Anktiva. Nab-Paclitaxel (100 mg/m²) was given in week 2, while week 3 comprised Nab-Paclitaxel; Anktiva (15 μg/kg), Sotevtamab (12 mg/kg), and Eflornithine (750 mg BID). In week 4, Sotevtamab was continued while Eflornithine was discontinued.

Cycle 2 began with Carboplatin, Nab-Paclitaxel, Anktiva, and Sotevtamab. Subsequent weekly treatments continued with Nab-Paclitaxel, Sotevtamab, and resumption of Eflornithine. Some doses (e.g., Nab-Paclitaxel in week 3 of cycle 2) were skipped due to low white blood cell counts.

Tumor Biomarker Response (CA-125) was monitored during the treatment. Initial CA-125 levels were 477 U/mL prior to therapy (normal <35 U/mL). After treatment cycles, CA-125 declined substantially, reaching 26.6 U/mL (normal level) after 7 weeks of treatment. This indicates a strong biochemical response to therapy. A CT scan after therapy initiation documented measurable treatment response, consistent with tumor shrinkage and clinical benefit.

Hematologic monitoring is shown in FIG. 9. Serial blood counts were tracked. White blood cells dropped as low as ˜2.4×10³/μL, hemoglobin decreased to ˜10.3 g/dL, and platelets fluctuated but remained above 114×10³/μL. These results are consistent with expected myelosuppressive effects of chemotherapy.

The patient's CA-125 reduction, imaging response, and maintained lymphocyte activity collectively support the present disclosure that the method of preconditioning a tumor microenvironment in vivo prior to resection or biopsy of a tumor as disclosed herein is not merely cytotoxic but reprograms the tumor microenvironment prior to surgery. Specifically: Chemotherapy (Carboplatin+Nab-Paclitaxel) debulks tumor and releases antigen; N-803 expands and activates NK/CD8⁺ effector cells; Sotevtamab promotes TLS for local immune priming; and Eflornithine remodels immunometabolic pathways, reducing suppression. Together, these effects unexpectedly and synergistically create an inflamed, antigen-rich, and lymphocyte-permissive TME that is optimally conditioned for immune recognition and long-term tumor control after resection.

### Example 3: Tumor Educated Lymphocyte (TEL) Production

Frozen PBMCs are thawed and cultured into a 24 well round bottom cell culture plate in XVIVO15 medium. The cells are treated with GM-CSF/FLT3L and either carcinoembryonic antigen (CEA) peptide pool consisting of overlapping peptides covering the entire protein (pepmix) or DMSO. On day 2, additional media containing human serum for a final 5% concentration and IL-7/IL-15/IL-21 are added. Media with human serum for a final concentration of 5% are added on day 4 and 7. The cells are then harvested on day 9, and restimulated with either the CEA pepmix or DMSO. T-cell activation is evaluated using intracellular flow cytometry using an extracellular antibody panel of CD3, CD4 and CD8, as well as an intracellular antibody panel of IFN-γ and TNF-α.

Following the expansion process, the CD8+ and CD4+ T-cells in the PBMCs educated with the CEA pepmix were able to demonstrate an IFN-γ response to CEA pepmix recall, but not when educated with DMSO. These results are shown in FIG. 12.

Thus, in this way, the inventors educated PBMC using CEA pepmix to achieve antigen-specific CD8+ and CD4+ recall responses. The same technique could be used to educate cells using full length protein, yeast lysate, or tumor lysates. For de novo antigen, this technique could test ability to generate SAR-COV2 or HIV response in naïve donors.

In some embodiments, the numbers expressing quantities of ingredients, properties such as concentration, reaction conditions, and so forth, used to describe and claim certain embodiments of the invention are to be understood as being modified in some instances by the term “about.” As used herein, the terms “about” and “approximately”, when referring to a specified, measurable value (such as a parameter, an amount, a temporal duration, and the like), is meant to encompass the specified value and variations of and from the specified value, such as variations of +/−10% or less, alternatively +/−5% or less, alternatively +/−1% or less, alternatively +/−0.1% or less of and from the specified value, insofar as such variations are appropriate to perform in the disclosed embodiments. Thus, the value to which the modifier “about” or “approximately” refers is itself also specifically disclosed. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. A method of treating a tumor in a patient in need thereof, the method comprising:
collecting an apheresis material from a whole blood sample of the patient;
isolating from the apheresis material at least two cell products selected from the group consisting of:
a T cell product, comprising γδ T cells, circulating tumor-educated lymphocytes (TELs), ex vivo educated TELs, γδ CAR-T cells, and αβ CAR-T cells;
an NK cell product, comprising invariant natural killer T (INKT) cells, NK cells, M-ceNK cells, ex vivo TEL INKT cells, CAR-NK cells, CAR-M-ceNK cells, and CAR-iNKT cells; and
a dendritic cell product, comprising educated dendritic cells; and
administering to the patient a composition comprising the at least two of the cell products to treat the tumor.

2. The method of claim 1, wherein the at least two cell products comprise the T cell product and the NK cell product.

3. The method of claim 1, wherein the at least two cell products comprise the T cell product and the dendritic cell product.

4. The method of claim 1, wherein the at least two cell products comprise the NK cell product and the dendritic cell product.

5. The method of claim 1, comprising three cell products and wherein the three cell products are the T cell product, the NK cell product, and the dendritic cell product.

6. The method of claim 1, wherein the NK cell product is iNKT cells.

7. The method of claim 1, further comprising administering an IL-15 superagonist.

8. A method of expanding ex vivo invariant natural killer T (INKT) cells, the method comprising:
isolating iNKT from a patient apheresis material intermediate (AMI); and
exposing the iNKT to an IL-15:IL-15Rα complex and a bacterial minicell derived nanoparticle, wherein the nanoparticle comprises alpha-galactosyl ceramide (αGC).

9. The method of claim 8, wherein the iNKT cells are bound to anti-iNKT microbeads and isolated by column chromatography.

10. The method of claim 9, wherein the microbeads are specific for the T cell receptor α-chain Vα24-Jα18.

11. The method of claim 8, wherein the microbeads are magnetic.

12. The method of claim 8, wherein the IL-15:IL-15Rα complex comprises N-803.

13. The method of claim 8, wherein the isolated iNKT cells are CD3+ and CD56+.

14. The method of claim 8, further comprising formulating the expanded iNKT cells for administration of an effective amount of the iNKT cells to a patient in need thereof.

15. A method of expanding ex vivo invariant natural killer T (INKT) cells, the method comprising exposing patient-derived apheresis material intermediate (AMI) to an IL-15:IL-15Rα complex and alpha-galactosyl ceramide (αGC).

16. The method of claim 15, wherein the αGC is stabilized by linkage to a protein.

17. The method of claim 15, wherein the αGC is stabilized by linkage to a bacterial minicell derived nanoparticle.

18-54. (canceled)

55. The method of claim 15, wherein the IL-15:IL-15Rα complex comprises N-803.

56. The method of claim 15, further comprising formulating the expanded iNKT cells for administration of an effective amount of the iNKT cells to a patient in need thereof.
